(19) United States
(12) Patent Application Publication
(10) Pub. No.: US 2010/0034894 A1
(43) Pub. Date: Feb. 11, 2010

(54) Use of Sucralose as a Granulating Agent

(76) Inventors: Christopher E. Szymczak, Marlton, NJ (US); Ryan Snyder, Colmar, PA (US); Kristin Costello, Phoenixville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/537,333

(22) Filed: Aug. 7, 2009

Related U.S. Application Data
(60) Provisional application No. 61/087,311, filed on Aug. 8, 2008.

(51) Int. Cl. A61K 9/16; A61K 47/36; A61K 31/138

(52) U.S. Cl. 424/490; 514/777; 514/658

(57) ABSTRACT

A method of making a granulation comprising the steps of (a) combining sucralose, a polar solvent, a wettable material and an active agent, thereby forming a mixture; and (b) drying the mixture, thereby forming the granulation.

### CROSS-REFERENCE TO RELATED APPLICATION

The present application claims the benefit of priority to U.S. Provisional Application Ser. No. 61/087,311, filed Aug. 8, 2008, the contents of which are completely incorporated by reference.

### BACKGROUND OF THE INVENTION

1. Field of the Invention

The present invention relates to solid dose compositions. More particularly, the present invention relates to solid dose compositions and the use of sucralose, an active agent, a polar solvent and at least one wettable material to make a granulation.

2. Related Background Art

For the purposes of granulating a powder (usually containing an active pharmaceutical), a granulating agent is traditionally added to the powder in order to increase the particle size of the powder. Increasing the particle size, and consolidating the particle into a more uniform size distribution improves the powder's flow characteristics, improves blending uniformity of active ingredients and makes it more compressible.

In addition, a granulated particle further facilitates coating using a fluidized bed coating process (Wurster, Rotor or Top Spray coating). A more uniform particle size distribution is desired for polymer particle coating since coating is used to taste-mask and/or control the release of the active ingredient.

Active ingredients are often incorporated into fast dissolving tablets or chewable tablets. The active ingredient can impart an undesirable bitter or burning attribute, in which case it is usually desirable to coat the active ingredient with an additional taste-masking coating.

However, tablets made in this manner have many undesirable attributes. For example, these gum based low calorie tablets have an unnatural mouth feel (e.g., slimmy, gummy, and/or thin), minimal aroma, and do not taste like natural tablets.

### SUMMARY OF THE INVENTION

The present invention is directed to a method of making a granulation comprising the steps of (a) combining sucralose, a polar solvent, a wettable material and an active agent, thereby forming a mixture; and (b) drying the mixture, thereby forming the granulation.

The present invention also includes a method of increasing the mean particle size of an active agent comprising the steps of combining sucralose, a polar solvent, a wettable material and the active agent, thereby forming a mixture; and drying the mixture, thereby forming a granule, wherein the mean particle size of the granule is at least about 1.0% greater than the mean particle size of the active agent.

In one particularly preferred embodiment, the present invention is a method of making a granulation composition comprising the steps of (a) coating/layering a wettable material with a solution or suspension comprising sucralose, a polar solvent, and an active agent, thereby forming a mixture; and (b) drying the mixture, thereby forming the granulation.

In another embodiment, the method comprises the steps of (a) combining sucralose, a polar solvent, a wettable material and an active agent, thereby forming a mixture; and (b) drying the mixture, thereby forming the granulation, wherein the granulation exhibits an increase in mean particle size of at least about 1.0% when compared to a substantially similar granulation composition absent the sucralose.

### DETAILED DESCRIPTION OF THE INVENTION

As used herein, “agglomeration” refers to a gathering together of particles into larger size units. The advantages of increasing the size of the powder lie in improving (i) the handling properties of the bulk materials, (ii) control over blend uniformity, (iii) compressibilty, (iv) the coating precision for coated granules, and (v) the flow of the dry material. The agglomeration process typically involves molecular bonding as well as a binding liquid. Numerous types of granulation and agglomeration processes are known. Common examples include compaction, extrusion, agitation, fusion, spray drying, high shear granulation and fluidized bed agglomeration.

Binders as used herein, are ingredients added to compounded dry powder mixtures of solids and the like to provide adhesive qualities during and after compression to make tablets or cakes. Many lipids, surfactants, and polymers can be used for the indicated purpose. The characteristics of a granulation are dependent upon several factors, including the materials used, the method of making the granulation, and the equipment. The binder is a component in the materials used and has a significant impact on the characteristics. For example, the uniformity of the granulation particle size, the hardness of the granule, the hardness of the final compressed tablet, the flowability of the granulation and compressibility.

Binders are either sugars or polymeric materials, such as natural polymers or synthetic polymers.

As used herein, “wettable material” refers to any powdered substance that will allow a part or whole droplet of a polar solvent to spread over its surface. Wettable materials may absorb or partially be solubilized by the polar solvent. A wettable material is further defined by analysis by use of a goniometer, wherein the contact angle is less than 90 degrees.

As used herein, “matrix” is defined as the portion of the tablet excluding the granulation.

As used herein, the “mean particle size” is defined by the geometric mean of the log-normal distribution of particles by weight in grams according to Martin's Physical Pharmacy, Chapter 16, Micrometrics, pp. 423-448, (Alfred Martin, 1993), which is incorporated herein by reference to simplify and demonstrate effect of the invention. Other methods known in the art of measuring particle size may be employed without limitation.

The present invention is directed to a method of making a granulation. The method includes the steps of (a) combining sucralose, a polar solvent, a wettable material and an active agent, thereby forming a mixture; and (b) drying the mixture, thereby forming the granulation.

It has been found that the use of sucralose during the granulation process increases the particle size of a granulation to a greater degree than without sucralose. The effect that the sucralose has on particle size growth can be demonstrated by making a granulation with sucralose in accordance to the present invention and comparing it to a the same granulation made without using sucralose. The sucralose can be used in either a wet or dry form. This novel effect of sucralose used in the inventive method has many advantages over the use of typical binding agents such as sugars, starches and cellulosic polymers that would traditionally be used to form granulations or an agglomerations of particles.

Furthermore, it has been discovered that the high adhesive strength formed between the wettable material and the active ingredients when using sucralose in a wet state is such that the granule does not return to its former particle size distribution after drying. Although it is known in the art that sucralose provides organoleptic sensory benefits, the use of sucralose as a binding agent with highly reactive compounds allows for the manufacture of novel dosage forms (such as chewable, dissolvable or other immediate release solid dosage forms) without adverse taste sensory characteristics found with some traditional binders or the formation of degradents after manufacture. Degradation pathways known in the art, which can degrade actives limits the use of traditional binding agents such as sugars, starches, glycols or cellulosic polymers. For example, some antihistamines with amine groups may become unstable and form degradation products in the presence of reducing sugars. Other active agents may be oxidized in the presence of glycols or cellulosic compounds.

Sucralose is chemically different from reducing sugars (such as sucrose or dextrose), cellulosic polymers, glycols and starches. It exhibits insignificant or no detectable reactivity in the examples previously mentioned in the normal course of product use. When used in the unique manner described by this invention, sucralose provides stable granulations which may be incorporated into nutritional or drug products. Thus, sucralose provides an alternative binding agent that is useful for manufacturing larger particles without having to use binding agents that may be reactive.

For the purposes of granulating a powder (e.g., an active pharmaceutical), a binding/granulating agent is traditionally added to the powder in order to increase the particle size of the powder. During fluid bed granulation or high shear granulation processes, this granulating agent is typically added to the bed of materials wherein a water based solution is sprayed onto the bed and dried. Alternatively, the granulating agent may be solubilized into solution and sprayed onto the bed of materials and dried. The bed of materials may include the active ingredient as well as other excipients, including but not limited to lubricants, fillers, compression aids, and additional binders. Increasing the particle size, and consolidating the particle into a more uniform size distribution makes the ingredient more flowable and compressible, and in addition, facilitates a fluidized bed particle coating process (e.g, Wurster, Rotor or Top Spray coating). A more uniform particle size distribution is desirable for polymer particle coating. A uniform particle size is desirable because it results in a coating having greater uniformity for taste-masking and/or modified release properties of the active ingredient in aqueous media.

Sucralose is known as a high intensity sweetener, for use in a wide variety of products including foods, beverages, liquid and solid pharmaceuticals and confectioneries. Typically, sucralose is dispersed into the matrix of a dosage form. In the present invention, sucralose is included as a component to assist in forming a granulation of an active ingredient (e.g., a pharmaceutical active agent). That is, in the present invention, sucralose serves as a binder in the production of particulates, including granules, granulations, and layered particle substrates.

The inventors have developed a method in which sucralose is used as a binding/granulating agent. In one embodiment sucralose is used as the sole binding/granulating agent. It has been found that in addition to its sweetening properties, sucralose can be used to bind the active ingredient into a granule, which aids in minimizing or eliminating the use of a traditional binding/granulating agent.

It has been found that the use of approximately 0.16 percent by weight (wt. %) of sucralose to active ingredient, e.g., dried granulation particles, results in an increase of at least about 1% in mean particle size versus granulation without the use of sucralose. Preferably, an increase of at least about 2%, more preferably an increase of at least about 3%, even more preferably an increase of at least about 5%, and still even more preferably an increase of at least about 8% in mean particle size is observed. The mean particle size is determined by measuring the distribution of particles in a sieve analysis across seven (7) sieves. Typical instruments used for determining particle size include, but are not limited to, an ATM Sonic sifter, which is commercially available from by the Sepor Corporation; as well as a FMC Sieve Shaker, which is commercially available from the FMC Corporation. Alternative methods of analyzing particle size include laser diffraction and light scattering devices, using analyzers such as a commercially available Horiba LA-950V2 Laser Diffraction Particle Size Analyzer, and a Horiba LB-550 Dynamic Light Scattering Particle Size Analyzer. Still further methods include camera based particle size analysis using analyzers such as a commercially available Horiba CAMSIZE Dynamic Image Analysis system, and accoustic spectroscopy methods using analyzers such as a commercially available Horiba DT-1201 Accoustic Spectroscopy Particle Size Analyzer. In a preferred embodiment, the method of the present invention produces an increase of at least about 2% of particle size, between 18 and 200 mesh sieves when using a sieve analysis method versus granulation without the use of sucralose. In another embodiment, the method of the present invention produces an increase of at least about 10% of particle size, between 50 and 60 mesh sieves when using a sieve analysis method versus granulation without the use of sucralose.

### Method of Making

The matrix tablet compositions of the present invention may be made by any method known to those skilled in the art so long as it results in a homogeneous mixture of the ingredients. Suitable methods include, for example, dry blending, spray drying, agglomeration, wet granulation, fluidized bed granulation, compaction, co-crystalization and the like. The granulation portion of the invention may be made by any granulation method known in the art where a polar solvent, such as water, is added to partially solubilize materials in the granulation.

Granulation is a process that forms a collection of particles together by creating bonds between them. There are several different methods of making a granulation. In tablet manufacturing, wet granulation is typically used. Alternatively, dry granulation methods may be used to form granules.

### Wet Granulation

In a wet granulation process, a binder or adhesive is incorporated into a liquid (e.g., granulating agent) and included in the powdered mixture in a rolling drum, which forms the agglomeration using agitation. Alternatively, the dry powdered binder is added to the active ingredient bed and the liquid in the form of polar solvents, such as water or an organic polar solvent, is added. Suitable organic polar solvents include but are not limited to ethanol, methanol, isopropanol and mixtures thereof. In one embodiment a mixture of water and an organic polar solvent is used. Granules are formed as the particles bind together. Bulk particles in the presence of a liquid binder or wetting agent are rolled into a semi-spherical or spherical shape depending on the type of process selected. The amount of liquid used should be properly managed to avoid overwetting or underwetting issues. Too much liquid leads to overwetting, which may result in granules that are (i) too large, (ii) too hard upon drying, or (iii) have a large particle size distribution. Conversely, too little liquid leads to underwetting, which causes the granules to be too soft and friable, or have a small particle size distribution. The solvent and powder mixture can form bonds between powder particles that are strong enough to lock them in together. After the solvent evaporates and the powders have formed a densely held mass, the granulation is milled which results in the formation of granules. For safety reasons, the use of aqueous solutions when permissible is preferred over other solvents.

A rolling drum is a form of agglomeration using agitation. Aggregates are formed by a snowball effect. Bulk particles in the presence of a liquid binder or wetting agent are rolled into a spherical shape.

Other forms of wet granulation processes include using high shear granulation and fluid bed drying or fluidized-bed granulation. Fluidized-bed granulation is a process performed in a vessel, where the powder is heated, granulated and dried on a bed of air. In the fluidized-bed process, aggregates are formed by the collision and coherence of fine particles and a liquid binder in a turbulent system. In the high shear process, the bed of materials is agitated using a mixing blade, and the wet liquid binder is added while mixing. The materials are then typically dried using fluid bed drying or tray drying. In one embodiment during high shear granulation the liquid comprises sucralose as a binding agent. In another embodiment, during high shear granulation, the bed includes sucralose and the liquid is slowly added to the bed. In another embodiment, the liquid includes sucralose and the bed contains an additional binding agent. In yet another embodiment, the liquid comprises sucralose and an additional binding agent. In still yet another embodiment, the bed contains sucralose and an additional binding agent. During high shear granulation processing, in one embodiment, sucralose is dissolved or suspended in the granulating liquid comprising sucralose, and a second active ingredient is contained in the bed.

In the fluidized bed granulation process, the liquid is sprayed onto the bed of materials, typically comprising the active ingredient and other excipients until the desired amount of liquid is added. The process is then switched into a drying mode where the granules are substantially dried using fluidized air. In one embodiment, the granulating liquid comprises sucralose as a binding agent. In another embodiment, the bed includes sucralose. In another embodiment, the granulating liquid comprises sucralose and the bed comprises an additional binding agent. In another embodiment, the liquid comprises sucralose and an additional binding agent. In another embodiment the bed comprises sucralose and an additional binding agent.

During fluidized bed processing, in one embodiment a first active ingredient is dissolved or suspended in the granulating liquid comprising sucralose, and a second active ingredient is contained in the bed.

In one preferred embodiment the active ingredient is dissolved in a polar solvent such as water and sprayed onto an wettable material such as microcrystalline cellulose in a fluid bed granulator. In one version of this embodiment, the following steps are carried out: (1) the active drug is dissolved in the solvent, (2) the microcrystalline celullose is blended with the sucralose in the fluid bed granulator, (3) the active drug solution is sprayed onto the solids mixture, wherein the sucralose facilitates binding to the wettable material, and (4) the layered particles are dried. In a second version of this embodiment the following steps are carried out (1) the active drug and sucralose are dissolved in the solvent, (2) the microcrystalline celullose is fluidized with the sucralose in the fluid bed granulator, (3) the active drug/sucralose solution is sprayed onto the solids mixture, wherein the sucralose facilitates binding to the wettable material and (4) the layered particles are dried. In this embodiment, the microcrystalline cellulose is the wettable material.

### Particle Coating

In one embodiment of the present invention the granules containing sucralose may be coated with a taste-masking or modified release coating. In addition to sucralose, the core of the granulated particle may comprise pure, crystalline active ingredient, or a mixture of active ingredient with optional ingredients, such as additional binders, surfactants, flavorants, sweeteners, release modifying agents, and other excipients known in the art. Suitable release modifying agents include but are not limited to polymers such as hypromellose, cellulose acetate, ethylcellulose, hydroxypropylcellulose, polyethylene oxides, and polymethacrylates. The average diameter of the coated particle may be from about 100 to about 400 microns, or about 150 to about 300 microns.

### Spray Drying

Spray drying is a method whereby a solution or slurry is rapidly dried into particulate form by atomizing the solution or slurry in a heated chamber. Typically, aqueous systems are used, but solvent-based systems may be used under controlled conditions. In the method of the present invention, the slurry comprises sucralose and at least one active ingredient, wherein the slurry is sprayed into a granule. In one embodiment the slurry may comprise additional excipients such as fillers, acidulants, flavors, lubricants, and additional active ingredients. In one embodiment the spray dried active ingredient is combined with a second active ingredient and compressed into tablets.

### Compaction

Another method that can be employed to form the core is by compressing the active agent and sucralose directly into tablets using a tablet press. “Compression,” as used herein, shall mean a process of forming a dosage form in a desired shape and size wherein a material is compacted into a tablet between the surfaces of punches via an increase in pressure before being removed therefrom.

The core of the coated particle may comprise any one of a number of active ingredients. Suitable active ingredients broadly include, but are not limited to, pharmaceutically active ingredients, dietary supplements, nutritionals, nutriceuticals, and the like. More specifically these include analgesics, decongestants, expectorants, antitussives, antihistamines, gastrointestinal agents, diuretics, proton-pump inhibitors, bronchodilators, sleep-inducing agents, vitamins, minerals, anti-infectives, nutrients, and mixtures thereof.

Tablets comprised of the particles of the present invention may be made by any means known in the art. Conventional methods for tablet production include direct compression (“dry blending”), dry granulation followed by compression, and wet granulation followed by drying and compression. Other methods include the use of compacting roller technology such as a chilsonator or drop roller, or molding, casting, or extrusion technologies. All of these methods are well known in the art, and are described in detail in, for example, Lachman, et al., “The Theory and Practice of Industrial Pharmacy,” Chapter 11, (3^{rd} Ed. 1986), which is incorporated by reference herein.

In one embodiment wherein the tablets are formed by the direct compression method, a blend of the particles having two coating layers, and any other appropriate optional ingredients are directly compacted. After blending, a pre-determined volume of particles is filled into a die cavity of a rotary tablet press, which continuously rotates as part of a “die table” from the filling position to a compaction position. The particles are compacted between an upper punch and a lower punch to an ejection position, at which the resulting tablet is pushed from the die cavity by the lower punch and guided to an ejection chute by a stationary “take-off” bar.

In embodiments wherein a chewable tablet is desired, the degree of particle compaction is controlled so that the resulting tablets are relatively soft, i.e., they have a hardness of up to about 15 kiloponds per square centimeter (kp/cm²). Preferably, from about 1 kp/cm² to about 10 kp/cm², and more preferably, from about 2 kp/cm² to about 6 kp/cm². “Hardness” is a term used in the art to describe the diametrical breaking strength as measured by conventional pharmaceutical hardness testing equipment, such as a Schleuniger Hardness Tester. In order to compare values across different size tablets, the breaking strength is normalized for the area of the break (which may be approximated as the tablet diameter times the thickness). This normalized value, expressed in kp/cm², is sometimes referred in the art as tablet tensile strength. A general discussion of tablet hardness testing is found in Leiberman et al., 2 Pharmaceutical Dosage Forms—Tablets, pp. 213-217 and 327-329 (2^{nd} Ed. 1990) (hereinafter “Lieberman”).

In one embodiment of the tablet described in the method of the present invention, a first quantity of sucralose is contained in the granulation composition and a second quantity of sucralose in contained in the compressed tablet matrix. In another embodiment, a second active ingredient may be present within the matrix of the tablet.

The chewable tablet may also contain other conventional ingredients within the matrix, such as fillers, including water soluble compressible carbohydrates such as dextrose, dextrose monohydrate, sucrose, mannitol, sorbitol, maltitol, xylitol, erythritol, lactose, and mixtures thereof, conventional dry binders including cellulose, cellulosic derivatives, polyvinyl pyrrolidone, starch, modified starch, and mixtures thereof, and in particular microcrystalline cellulose; sweeteners including aspartame, acesulfame potassium, sucralose and saccharin; disintegrants such as microcrystalline cellulose, starch, sodium starch glycolate, crosslinked polyvinylpyrrolidone, crosslinked carboxymethylcellulose; and lubricants, such as magnesium stearate, stearic acid, talc, and waxes. The chewable tablet may also incorporate pharmaceutically acceptable adjuvants, including for example preservatives, flavors, acidulants, antioxidants, glidants, surfactants, and coloring agents.

In one embodiment, the method of the present invention includes blending the coated active ingredient comprising a granule with a first quantity of sucralose into a matrix comprising dextrose monohydrate and a second quantity of sucralose. The dextrose monohydrate is present in the tablet in directly compressible form. That is, the dextrose monohydrate has an average particle size of about 100 to about 500 microns, preferably about 100 to about 250 microns, and more preferably about 150 to about 200 microns. Such a particle size is required to impart the formulation with adequate flowability and compressibility, and with a smooth and creamy mouthfeel according to the invention. The amount of dextrose monohydrate in the tablet is typically about 15 to about 90% by weight, preferably about 25 to about 85% by weight, and more preferably about 30 to about 75% by weight of the total weight of the tablet.

### Co-Crystallization

In a co-crystallization process, a supersaturated solution is formed and co-crystallization agents are introduced. The mixture is then subjected to conditions that either spontaneously produce crystals or alternatively, the mixture is seeded with crystals of the desired substance to produce crystals.

Optionally, the method may include a coating step. The coating may be applied to mask the taste of the active agent using a taste-masking or modified release polymer system. In addition, the coating protects the core and tablet from temperature and humidity constraints. Typically, sugar and film coatings are applied to the tablets.

Alternatively, the core granules may be compressed into tablets using tablet presses.

In one embodiment, sucralose is included in the granulating or drug layering solution. In another embodiment, the concentration of the sucralose in a solution comprising a polar solvent is from about 0.01% to about 30% by weight, preferably, from about 0.05% to about 10%, and more preferably from about 0.1% to about 10%.

In another embodiment, sucralose is included in a powder bed containing the active ingredient and water, or a polymer solution is sprayed into the granulation and dried.

The present invention includes a method of increasing the particle size of a core granule comprising the step of including about 0.01 to about 5 wt. % sucralose with an active agent and a wettable material, by weight of the granulation, wherein the particle size of the granule increases by at least about 2 wt. % as measured by the weight of material through an 18 mesh screen and retained on a 200 mesh screen using sieve analysis, versus the particle size of the materials prior to granulation, including the mixture of the active agent and the wettable material.

The present invention also includes a composition made by the process comprising the step of forming a core comprising an active agent and sucralose.

In one particular embodiment, the present invention is a pharmaceutical composition comprising a core consisting essentially of an active agent and sucralose.

Optionally, the method may include the step of coating the core composition. The coating may be applied using any means that would provide a uniform taste-masked or modified release coated particle. In one embodiment, a modified release coating is applied so that it prevents or retards the release of the active ingredient. The coating may be a polymeric film forming polymer and may contain emulsifiers, plasticizers, surfactants, lubricants, and/or other ingredients.

In a preferred embodiment, the granulation portion of the tablet composition has a moisture content (on a weight percentage basis) of at least about 0.01%, preferably, less than about 5.0%. Alternatively, the moisture content of the granulation portion is about 0.05% to about 1.0%, more preferably, about 0.05% to about 0.8%, and even more preferably, about 0.1% to about 0.5%.

### Active Agent

In one embodiment, the active ingredient is a pharmaceutical active ingredient. The active ingredient is present in a safe and effective amount, which means an amount of the agent that is high enough, when administered orally, to significantly positively modify the condition to be treated or prevent an adverse or unwanted condition through short-term immediate use or repeated long-term chronic use within the scope of sound medical judgment. The safe and effective amount of the active agent will vary with the particular condition being treated; the physical condition and age of the patient being treated; the nature of concurrent therapy, if any; the duration of the treatment; the particular carrier utilized; the specific active agent(s) employed; and the like.

Typically, the active agent(s) are used in an amount, based upon the total weight of the granule composition, from about 45% to about 99%, e.g., from about 30% to about 70%. In cases where the granule is coated, the active agent, based on the total weight of the coated particles is from about 25% to about 65%, e.g. from about 30% to about 60%.

The active agents useful herein can be selected from classes from those in the following therapeutic categories: ace-inhibitors; alkaloids; antacids; analgesics; anabolic agents; anti-anginal drugs; anti-allergy agents; anti-arrhythmia agents; antiasthmatics; antibiotics; anticholesterolemics; anticonvulsants; anticoagulants; antidepressants; antidiarrheal preparations; anti-emetics; antihistamines; antihypertensives; anti-infectives; anti-inflammatories; antilipid agents; antimanics; anti-migraine agents; antinauseants; antipsychotics; antistroke agents; antithyroid preparations; anabolic drugs; antiobesity agents; antiparasitics; antipsychotics; antipyretics; antispasmodics; antithrombotics; antitumor agents; antitussives; antiulcer agents; anti-uricemic agents; anxiolytic agents; appetite stimulants; appetite suppressants; beta-blocking agents; bronchodilators; cardiovascular agents; cerebral dilators; chelating agents; cholecystekinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypoglycemic agents; ion-exchange resins; laxatives; migraine treatments; mineral supplements; mucolytics, narcotics; neuroleptics; neuromuscular drugs; nutritional additives; peripheral vasodilators; polypeptides; prostaglandins; psychotropics; renin inhibitors; respiratory stimulants; sedatives; steroids; stimulants; sympatholytics; thyroid preparations; tranquilizers; uterine relaxants; vaginal preparations; vasoconstrictors; vasodilators; vertigo agents; vitamins; wound healing agents; and others.

One class of preferred active ingredients include nonsteroidal anti-inflammatory drugs (NSAIDs), such as ibuprofen, ketoprofen, flurbiprofen, naproxen, diclofenac, rofecoxib, celecoxib, and aspirin. The active ingredient may alternatively be selected from acetaminophen, pseudoephedrine, phenylpropanolamine, chlorpheniramine, dextromethorphan, diphenhydramine, dimenhydrinate, meclizine, famotidine, loperamide, ranitidine, cimetidine, bisacodyl, psyllium, astemizole, loratadine, desloratadine, fexofenadine, cetirizine, antacids, mixtures thereof and pharmaceutically acceptable salts or metabolites thereof Most preferably, the active ingredient is selected from the group consisting of aspirin, acetaminophen, ibuprofen, pseudoephedrine, dextromethorphan, diphenhydramine, chlorpheniramine, loratadine, calcium carbonate, magnesium hydroxide, magnesium carbonate, magnesium oxide, aluminum hydroxide, mixtures thereof, and pharmaceutically acceptable salts thereof

Examples of suitable gastrointestinal agents include, but are not limited to, antacids such as calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof, H2 receptor antagonists, such as famotidine, ranitidine, cimetadine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate and loperamide; glycopyrrolate; antiemetics, such as ondansetron, analgesics, such as mesalamine.

In another embodiment of the invention, the active ingredient may be selected from pseudoephedrine, phenylephrine, phenylpropanolamine, chlorpheniramine, dextromethorphan, diphenhydramine, guaifenesin, astemizole, terfenadine, chlophedianol, fexofenadine, loratadine, desloratidine, doxilamine, menthol, norastemizole, cetirizine, benzocaine mixtures thereof and pharmaceutically acceptable salts, esters, isomers, and mixtures thereof.

In another embodiment, the active ingredient may be methylphenidate, modafinil and other active agents suitable for attention deficit hyperactivity disorder or attention deficit disorder, oxybutynin, sidenafil, and pharmaceutically acceptable salts, esters, isomers, and mixtures thereof.

Active agents may further include, but are not limited to food or herbal extracts; insoluble metal and mineral hydroxides, carbonates, oxides, polycarbophils, and salts thereof, adsorbates of active drugs on a magnesium trisilicate base and on a magnesium aluminum silicate base, and mixtures thereof.

In another embodiment, the active ingredient may be a nutraceutical. The term “nutraceutical” is understood to refer to food extracts and derivatives that are believed to have a beneficial effect on human health. The nutraceutical is usually contained in a medicinal format such as a capsule, tablet or powder in a prescribed dose.

Nutraceutical implies that the extract or food is demonstrated to have a physiological benefit or provide protection against a chronic disease.

Functional foods are defined as being consumed as part of a usual diet but are demonstrated to have physiological benefits and/or reduce the risk of chronic disease beyond basic nutritional functions.

Examples of claims made for nutraceuticals are resveratrol from red grape products as an antioxidant, soluble dietary fiber products, such as psyllium seed husk for reducing hypercholesterolemia, broccoli (sulforaphane) as a cancer preventative, and soy or clover (isoflavonoids) to improve arterial health. Such claims are being researched and many citations are available via PubMed to ascertain their foundation of basic research.

Other nutraceutical examples are flavonoids antioxidants, alpha-linolenic acid from flax seeds, beta-carotene from marigold petals, anthocyanins from berries, etc. With the US Dietary Supplement Health and Education Act (DSHEA), several other compounds were added to the list of supplements originally mentioned in FDA notification. Thus, many botanical and herbal extracts such as ginseng, garlic oil, etc. have been developed as nutraceuticals.

Nutraceuticals are often used in nutrient premixes or nutrient systems in the food and pharmaceutical industries.

Functional food or medicinal food is any fresh or processed food claimed to have a health-promoting and/or disease-preventing property beyond the basic nutritional function of supplying nutrients, although there is no consensus on an exact definition of the term.

Functional foods are sometimes called nutraceuticals, a blend of the words nutrition and pharmaceutical, and can include food that has been genetically modified. The general category includes processed food made from functional food ingredients, or fortified with health-promoting additives, like “vitamin-enriched” products, and also, fresh foods (e.g., vegetables) that have specific claims attached. Fermented foods with live cultures are often also considered to be functional foods with probiotic benefits.

Any of the active agents set forth above, pharmaceutically acceptable salts thereof, pharmaceutically acceptable enantiomers thereof, and mixtures thereof are also suitable for use in the present invention.

The active agent is included in the tablet composition in an amount from about 0.05 wt. % to about 30 wt. %, based on the total weight of tablet composition. Preferably, the active agent is about 0.1 wt. % to about 20 wt. %, and more preferably, about 0.5 wt. % to about 10 wt. %, based on the total weight of the tablet composition.

The active agent may be present in the dosage form in a variety of forms. For example, the active agent(s) may be dispersed at the molecular level, e.g., melted or dissolved, within the dosage form, or they may be in the form of particles, which in turn may be coated or uncoated. If the active ingredient is in form of particles, the particles (whether coated or uncoated) typically have an average particle size of about 1 micron to about 2000 microns. In one embodiment, such particles are crystals having an average particle size of about 1300 microns. In another embodiment, the particles are granules or pellets having an average particle size of about 50 microns to about 2000 microns, for example about 50 microns to about 1000 microns or from about 100 microns to about 800 microns.

### Sucralose

High intensity sweeteners are well known alternatives to nutritive sweeteners. They provide sweetness without the calories and other metabolic impacts of the nutritive sweeteners. In many cases, high intensity sweeteners provide a sweet flavor that is preferred to nutritive sweeteners. Some high intensity sweeteners, such as, aspartame, are nutritive, but are so intense that they still provide negligible calories because very small amounts are required. Other high intensity sweeteners, such as, for example, sucralose, are not absorbed when ingested and are, therefore, non-nutritive sweeteners.

Sucralose is known as a high intensity sweetener, for use in a wide variety of products including foods, beverages, liquid and solid pharmaceuticals and confectioneries. In most cases sucralose is dispersed into the matrix of the dosage form. In the case of this invention sucralose is added to the granulation of an active pharmaceutical ingredient.

Sucralose, which is also known as 4,1,6′-trideoxy-galactosucrose, is a heat-stable, high-intensity sweetener that may be produced in accordance with the process disclosed in U.K. Patent No. 1,543,167, and U.S. Pat. Nos. 5,136,031 and 5,498,709, which are incorporated by reference herein.

Sucralose may be included as either a dry component or as a liquid solution component. When sucralose is included as a dry component in the granulation prior to the addition of a solvent, it is essential that the moisture content of the active ingredient, the wettable material and the sucralose be increased during the granulation step (i.e., prior to drying) to at least about 0.01% by weight above the moisture content of the dry mixture alone to a maximum of about 30% moisture, e.g. from about 0.05 to about 10% moisture, by weight of the entire mixture including the added water.

The sucralose is present in an amount from about 0.01 weight percent (wt. %) to about 5.0 wt. %, based on the total weight of the granulation composition. Preferably, the sucralose is about 0.05 wt. % to about 0.5 wt. %, more preferably, about 0.09 wt. % to about 0.50 wt. %, and most preferably, about 0.10 wt. % to about 0.30 wt. %, based on the total weight of the granulation composition.

In embodiments where the granulation of the present composition is coated with a polymer system, the sucralose is about 0.05 wt. % to about 0.5 wt. %, more preferably, about 0.07 wt. % to about 0.30 wt. %, and most preferably, about 0.10 wt. % to about 0.20 wt. %, based on the total weight of the coated granulation.

In the granulation composition, the ratio on a weight basis of the active ingredient to sucralose is about 6.25:0.005 to about 6.25:0.05. Preferably, the ratio is about 6.25:0.01 to about 6.25:0.03, and most preferably, about 6.25:0.015 to about 6.25:0.025.

The sucralose is present in an amount from about 0.001 wt. % to about 0.05 wt. %, based on the total weight of the tablet composition. Preferably, the sucralose is about 0.001 wt. % to about 0.01 wt. %, more preferably, about 0.002 wt. % to about 0.01 wt. %, and most preferably, about 0.003 wt. % to about 0.008 wt. %, based on the total weight of the tablet composition.

In one embodiment the granulation particles containing sucralose as a binder are blended with a matrix in order to create a chewable tablet or an orally dissolving tablet. The granulation containing sucralose is prepared to more closely match the particle size of the matrix in order to uniformly blend the tablet blend (i.e., for blend uniformity), and to match the texture of the remaining matrix materials in order to obtain beneficial organoleptic properties. In one embodiment, the active granulation is less than about 25%, preferably, less than about 10% of the weight of the chewable tablet. In one embodiment, the weight ratio of the matrix materials in the tablet blend to the granulation containing sucralose is from about 75:25 to about 98:2.

### Wettable Material

In one embodiment, a wettable material may be included with the wettable material prior to the addition of the active ingredient in the method of the present invention. Typically, the wettable material may be present when a drug layering process is used to form the agglomerated particles. Drug layering has the advantage of using a material with a uniform particle size and is able to maintain that uniformity when spraying on the active ingredient. Suitable inert substrates include but are not limited to, dextrose, dextrose monohydrate, microcrystalline cellulose, spherical microcrystalline cellulose and mixtures thereof. In one embodiment, the active ingredient is dissolved into a liquid and sprayed into a bed comprising microcrystalline cellulose and sucralose.

The wettable material may be included in the method of the pharmaceutical composition in an amount from about 25 wt. % to about 75 wt. %, based on the total weight of the granulation composition. Preferably, the wettable material is about 35 wt. % to about 65 wt. %, and more preferably, about 45 wt. % to about 55 wt. %, based on the total weight of the granulation composition.

In embodiments where the granulation is coated, the wettable material may be included in the coated granulation in an amount by weight of the coated granulation from about 20 wt. % to about 60 wt. %, based on the total weight of the coated granulation composition. Preferably, the wettable material is about 20 wt. % to about 45 wt. %, and more preferably, about 30 wt. % to about 40 wt. %, based on the total weight of the coated granulation composition.

The wettable material may be included in the pharmaceutical composition in an amount from about 0.05 wt. % to about 15 wt. %, based on the total weight of the tablet core composition. Preferably, the wettable material is about 1 wt. % to about 5 wt. %, and more preferably, about 1 wt. % to about 3 wt. %, based on the total weight of the tablet core composition.

The active agent is applied to the wettable material by any conventional techniques known in the industry. For example, pan coating, roto-granulation, or fluidized bed layering. During such coating operations, the active agent is dissolved or dispersed in a solvent.

### Polar Solvents

Polar Solvents for use in the method of the present invention include aqueous and organic polar solvents. In one preferred embodiment, the polar solvent is water. Suitable organic polar solvents include, but are not limited to, ethanol, methanol, isopropanol and mixtures thereof. In one embodiment, a mixture of water and an organic polar solvent is used. In another embodiment, a polar solvent is a single or multi-component liquid with a dielectric constant greater than 24 where pure water has a measured dielectric constant of 80 and ethanol has a dielectric constant of 25.3 at 293.2K.

### Binders

Optionally, the granulation composition of the present invention may include additional binders.

During the granulation step, typical additional granulating agents are known as binders and are selected from polymers such as hypromellose, polyvinylpyrrolidone (PVP), hydroxypropylcellulose, starches such as cornstarch and pregelatinized starch, and modified starches.

Granulating agents may be added to a granulating solution in a solubilized or suspended state. Alternatively, granulating agents may be added to the powder blend, where water is sprayed onto the powder bed, causing partial solubilization of the granulating agent and subsequent bridging of the active ingredient, the granulating agent and any other optional excipients.

Binders are ingredients added to compounded dry powder mixtures of solids and the like to provide adhesive qualities during and after compression to make tablets or cakes. Many lipids, surfactants, and polymers can be used for the indicated purpose. The following list is limited essentially to ingredients frequently used as binders.

The characteristics of a granulation are dependent upon several factors, including the materials used, the method of making the granulation, and the equipment. The binder is a component in the materials used and has a significant impact on the characteristics. For example, the uniformity of the granulation particle size, the hardness, and compressibility.

Binders are either sugars or polymeric materials, such as natural polymers or synthetic polymers.

TABLE X
Typical Binders Used in Wet Granulation

| BINDER | Typical PERCENTAGE for use |
|---|---|
| Starch | 5-10% w/v aqueous paste |
| Pregelatinized Starch | 5-10% added dry to powder |
| Gelatin | 2-10% aqueous solution or 2% in |
| | starch paste |
| Polyvinylpyrrolidone | 5-20% aqueous or alcoholic solution |
| Methylcellulose (various | 2-10% aqueous solution |
| viscosity grades) | |
| Sodium carboxymethylcellulose | 2-10% aqueous solution |
| (low viscosity grade) | |
| Ethylcellulose (various | 5-10% alcohol or hydroalcoholic |
| viscosity grades) | solution |
| Polyacrylamides (Polymer JR) | 2-8% aqueous solution |
| Polyvinyloxoazolidone (Devlex) | 5-10% aqueous or hydroalcoholic |
| | solution |

Similarly, an organic acid may be included in the granule composition in an amount from about 0.5 wt. % to about 40 wt. %, based on the total weight of the granulation composition. Preferably, the acid is about 1.0 wt. % to about 30 wt. %, and more preferably, about 1.0 wt. % to about 10 wt. %, based on the total weight of the granulation composition. Suitable organic acids include but are not limited to fumaric, tartaric, citric, and malic acids.

In some cases it may be desirable to omit the additional wet binder. Certain binders can cause reactions with active ingredients where they may degrade, or they contain impurities, which cause reactivity with certain active ingredients (i.e. polyvinylpyrrolidone may contain peroxides). In one embodiment the granulation is substantially free of an additional wet binder. As used herein substantially free includes less than 0.5% or less than 0.1% by weight of the granulation.

### Optional Components

Optionally, a variety of ingredients may be included in the matrix of the tablet composition of the present invention.

Any coloring agent suitable for use in a food or pharmaceutical product may be used in the present invention and may include, but not be limited to azo dyes, quinopthalone dyes, triphenylmethane dyes, xanthene dyes, indigoid dyes, iron oxides, iron hydroxides, titanium dioxide, natural dyes, and mixtures thereof More specifically, suitable colorants include, but are not limited to patent blue V, acid brilliant green BS, red 2G, azorubine, ponceau 4R, amaranth, D&C red 33, D&C red 22, D&C red 26, D&C red 28, D&C yellow 10, FD&C yellow 5, FD&C yellow 6, FD&C red 3, FD&C red 40, FD&C blue 1, FD&C blue 2, FD&C green 3, brilliant black BN, carbon black, iron oxide black, iron oxide red, iron oxide yellow, titanium dioxide, riboflavin, carotenes, antyhocyanines, turmeric, cochineal extract, clorophyllin, canthaxanthin, caramel, betanin, and mixtures thereof.

Similarly, an organic acid may be included in the tablet composition in an amount from about 0.1 wt. % to about 20 wt. %, based on the total weight of the tablet composition. Preferably, the acid is about 0.1 wt. % to about 2 wt. %, and more preferably, about 0.25 wt. % to about 0.75 wt. %, based on the total weight of the tablet composition. Suitable organic acids include but are not limited to fumaric, tartaric, citric, and malic acids.

The compositions can contain other components, including flavor, aroma, other nutritional component, binders, and mixtures thereof

### Properties or Characteristics

In one embodiment, the strength of the granule is measured by the hardness of the granule. In another embodiment, the strength of the granule is measured using texture analysis as a measure of force. The granule sample is placed beneath a metal force probe such as a compression plate on a texture analyzer, such as a model TA-XT2i (HR) available from Texture Technologies Corporation, which crushes the granule from the surface and determines the force value at break, as well as the maximum force over time in a measurement of grams, milliNewtons or Newtons. In order to determine the force value, a granulation using sucralose according to the method of the present invention may be prepared and compared to a granule of a similar size prepared by the same method without the inclusion of sucralose. In one embodiment, the force value is at least 1% greater in a granule sample containing sucralose versus a sample without sucralose.

Another method of analyzing granules involves placing the granules into a vibrating container for a specified period of time to determine the level of undamaged granules, as indicated in U.S. Pat. No. 6,133,601, which is incorporated herein by reference. In one embodiment, the mass of undamaged granules as a fraction of the total mass when using a 30 mg sample of a granulation of the invention is at least 1% greater than the level of a 30 mg sample of a typical granulation, which does not contain sucralose and is prepared according to the same method.

### EXAMPLES

### Example 1

### Comparative

### Part A: Preparation of Drug Layering Solution Comprising Diphenhydramine

63.3 kg of purified water was added to a suitable stainless steel solution tank. A LIGHTNIN® Mixer was positioned in the tank so the mixing element/propeller was submerged in the water and the mixing speed was adjusted to create a vortex. 80.6 kg of diphenhydramine hydrochloride was added and mixed for approximately 1 hour. The solution was allowed to stand and deaerated for approximately 30 minutes.

### Part B: Layering, Drying and Sieving of Layered Diphenydramine Particles Without Sucralose

74.4 kg of Microcrystalline Cellulose (AVICEL® PH 200) were vacuum charged into a Glatt R-1400 Rotary Fluid Bed Granulating/Coating Unit. 134.2 kg of the aqueous solution containing diphenhydramine from Part A was then sprayed onto the AVICEL® PH 200 at an inlet air temperature of 55-60° C. and an inlet air flow of 895-1200 sCFM, a Rotor speed of 70 to 100 RPM, an atomization air pressure of 4 bars, and a solution spray rate of 660 g/minute for 25 kg of solution, 830 g/minute for 25 kg of solution, and 1030 g/minute for 84.2 kg of solution in three separate steps. The drug-layered AVICEL® was then dried at 65° C. and 1800 sCFM, discharged and screened through a vibratory screen separator equipped with an 18-mesh screen. A theoretical yield of 150.0 kg was anticipated, with 50.0% microcrystalline cellulose and 50.0% diphenhydramine, and by weight of the layered diphenhydramine particles.

A particle size analysis was performed using a vibratory shaker equipped with stainless steel vibratory screens. The batch demonstrated a mean particle size of 247 microns, a standard deviation=1.3 1, with particles having a ±1 standard deviation between 181 and 324 microns, with the following individual screen measurements:

| Mesh Size | % Retained |
|---|---|
| 30 Mesh | 0.10 |
| 40 Mesh | 1.35 |
| 50 Mesh | 19.92 |
| 60 Mesh | 28.72 |
| 80 Mesh | 39.17 |
| 100 Mesh | 7.16 |
| PAN | 3.58 |

### Part C: Preparation of Taste-Masking Coating Solution

552.2 kg of Acetone was added to a suitable stainless steel mixing tank. The LIGHTNIN® mixing blade was adjusted to be submerged in the tank. 58.3 kg of cellulose acetate and 3.1 kg of basic polymethacylate (EUDRAGIT® E100) was weighed and placed into a hopper. The hopper slowly augered the polymers into the acetone while mixing, and was mixed for approximately 120 minutes. The cellulose acetate and EUDRAGIT® E100 were prepared in a ratio of 95:5 and the solution was prepared as a 10% solids solution.

### Part D: Coating of Diphenhydramine Particles Without Sucralose

135.0 kg of the layered diphenhydramine particles from Example 1, Part A were vacuum charged into the Glatt granulating unit described in Example 1, Part B. The taste-masking coating solution from Part C was sprayed onto the particles utilizing an inlet air temperature of 50° C., a process air flow of 2484 sCFM, a rotor speed of 300 RPM, and a solution spray rate of 750-1500 RPM in multiple spray steps. The particles were then dried at an inlet air temperature of 62° C. until a product temperature of 60° C. was achieved. A coating level of about 30% when calculated by weight of the final coated particles was added. The particles were then discharged and sieved through an 18 mesh screen.

### Example 2

### Diphenhydramine Particles Comprising Sucralose as a Binder

### Part A: Preparation of Drug Layering Solution Comprising Diphenhydramine and Sucralose

63.3 kg of purified water was added to a suitable stainless steel solution tank and the LIGHTNIN® Mixer shaft was adjusted to be submerged in the water and the air pressure for regulating mixing speed was adjusted to obtain a vortex. 80.6 kg of diphenhydramine hydrochloride and 0.3 kg (300 grams) of sucralose powder were added and mixed for approximately 1 hour. The solution was then allowed to stand and deaerate for approximately 30 minutes. The viscosity of the solution when tested using a Zahn Cup #2 is between 20 and 25 seconds.

Part B: Layering, Drying and Sieving of Diphenydramine Particles with Sucralose

74.4 kg of Microcrystalline Cellulose (AVICEL® PH 200) were vacuum charged into the Glatt R-1400 Rotary Fluid Bed Granulating/Coating Unit. 134.5 kg of the aqueous solution containing diphenhydramine from Example 2, Part A was then sprayed onto the AVICEL® PH 200 at an inlet air temperature of 55-60° C., an inlet air flow of 1200-1800 sCFM, a Rotor speed of 70 to 100 RPM, an atomization air pressure of 4 bars, and a solution spray rate of 630 g/minute for 25 kg of solution, 800 g/minute for 25 kg of solution, and 1000 g/minute for 84.2 kg of solution in three separate steps. The drug-layered AVICEL® was then dried at 65° C. and 1800 sCFM, discharged and screened through a vibratory screen separator equipped with an 18-mesh screen. A theoretical yield of 150.0 kg was anticipated, with 49.9% microcrystalline cellulose, 49.9% diphenhydramine, and 0.2% of sucralose by weight of the layered diphenhydramine particles.

A particle size analysis was performed using a vibratory shaker equipped with stainless steel vibratory screens. The batch demonstrated a mean particle size of 270 microns, a standard deviation=1.24, with particles having a ±1 standard deviation between 218 and 336 microns with the following individual screen measurements:

| Mesh Size | % Retained |
|---|---|
| 30 Mesh | 0.00 |
| 40 Mesh | 1.56 |
| 50 Mesh | 33.85 |
| 60 Mesh | 31.88 |
| 80 Mesh | 30.25 |
| 100 Mesh | 1.90 |
| PAN | 0.54 |

### Part C: Preparation of Taste-Masking Coating Solution

552.2 kg of Acetone was added to a suitable stainless steel mixing tank. The LIGHTNIN® mixing blade was adjusted to be submerged in the tank. 58.3 kg of cellulose acetate and 3.1 kg of basic polymethacylate (EUDRAGIT® E100) was weighed and placed into a hopper. The hopper slowly augered the polymers into the acetone while mixing, and was mixed for approximately 120 minutes. The cellulose acetate and EUDRAGIT® E100 were prepared in a ratio of 95:5 and the solution was prepared as a 10% solids solution.

Part D: Coating of Diphenhydramine Particles with Sucralose

135.0 kg of the layered diphenhydramine particles from Example 2, Part A were vacuum charged into the Glatt granulating unit described in Example 2, Part B. The taste-masking coating solution from Part C was sprayed onto the particles utilizing an inlet air temperature of 50° C., a process air flow of 2484 sCFM, a rotor speed of 300 RPM, and a solution spray rate of approximately 750-1500 RPM in multiple spray steps. A coating level of about 30% when calculated by weight of the final coated particles was added. The particles were then dried at an inlet air temperature of 62° C. until a product temperature of 60° C. was achieved. The particles were then discharged and sieved through an 18 mesh screen.

### Example 3

### Basic Granulations Utilizing Sucralose

Granulations were produced using sucralose and microcrystalline cellulose to evaluate the impact of different levels of sucralose on the resulting particle size. Two grades of microcrystalline cellulose were used, which are commercially sold by the FMC Corporation under the brand names of AVICEL® pH 105 and AVICEL® pH 102. Approximately 350 grams of AVICEL® was used for each batch experiment. For batches using AVICEL® pH 105, 254.3 g of purified water was added. For batches using AVICEL® pH 102, 255.7 g of purified water was added.

### Part A: AVICEL® pH 105 Batches

### Sample 1A (Dry Screened):

As a control, 254.3 g of purified water was slowly added manually to 350 g AVICEL® over 25-35 minutes while mixing in a 2-quart Hobart mixer. The mixture was dried at 50° C. for 24 hours and screened though a 20 mesh screen.

### Samples 1.1B, 1.2B, 1.3B, 1.4B (Dry Screened):

0.01, 0.05, 0.1, and 1% of sucralose respectively was prepared as four solutions in 254.3 g of water per solution. Each granulation sample was prepared by slowly and manually adding the individual sucralose solutions to 350 g of AVICEL® while mixing over 25-35 minutes in a 2-quart Hobart mixer. The samples were dried at 50° C. for 24 hours and screened though a 20 mesh screen.

### Samples 1.1C, 1.2C, 1.3C, 1.4C (Dry Screened):

0.01, 0.05, 0.1, and 1% of sucralose respectively was blended individually with 350 g of AVICEL® each as a dry mixture in a 2-quart Hobart mixing bowl. 254.3 g of water was slowly and manually added to each sample while mixing over 25-35 minutes in a 2-quart Hobart mixer. The samples were dried at 50° C. for 24 hours and screened though a 20 mesh screen.

### Sample 1D (Wet Screened):

As a control, 254.3 g of purified water was slowly added manually to 350 g AVICEL® over 25-35 minutes while mixing in a 2-quart Hobart mixer. The mixture was screened though a 20 mesh screen, and then dried at 50° C. for 24 hours.

### Samples 1.1E, 1.2E (Wet Screened):

0.01% and 5% of sucralose respectively, was prepared as two solutions in 254.3 g of water per solution. Each granulation sample was prepared by slowly and manually adding the individual sucralose solutions to 350 g of AVICEL® while mixing over 25-35 minutes in a 2-quart Hobart mixer. The samples were screened though a 20 mesh screen and then dried at 50° C. for 24 hours.

### Samples 1.1F, 1.2F (Wet Screened):

0.01 and 5% of sucralose respectively was blended individually with 350 g of AVICEL® each as a dry mixture in a 2-quart Hobart mixing bowl. 254.3 g of water was slowly and manually added to each sample while mixing over 25-35 minutes in a 2-quart Hobart mixer. The samples were screened though a 20 mesh screen and dried at 50° C. for 24 hours.

The particle size results for batches produced in Part A (using AVICEL® pH 105) are displayed in Table 1. Particle size was analyzed via sieve cut analysis, using an ATM Sonic Sifter and approximately 10 g of granulation. The amounts of material retained on each sieve cut are displayed in Table 2 and Table 3. The results demonstrate that the addition to of sucralose to the solution and in the dry blend results in a substantial increase in particle size, demonstrating the binding effect. The range of particle size increase for particles greater than 74 microns (200 mesh) was between 3.7 and 27.8%. These results are evident both in wet screening and screening of the material following a drying step.

TABLE 1

| | | | | |
|---|---|---|---|---|
| | % | % >74 | Mean Particle | >74 microns |
| SAMPLE | SUCRALOSE | microns | Size (microns) | % Increase |

Control, Screened dry (No Sucralose)

| | | | |
|---|---|---|---|
| 1A | 0.00 | 46.91 | 73 |

Sucralose Added to water, Screened dry

| | | | | |
|---|---|---|---|---|
| 1.1B | 0.01 | 58.59 | 95 | 24.9 |
| 1.2B | 0.05 | 59.96 | 102 | 27.8 |
| 1.3B | 0.10 | 58.17 | 102 | 24.0 |
| 1.4B | 1.00 | 57.34 | 100 | 22.2 |

Sucralose to the bowl dry, Screened dry

| | | | | |
|---|---|---|---|---|
| 1.1C | 0.01 | 54.77 | 93 | 16.8 |
| 1.2C | 0.05 | 57.65 | 99 | 22.9 |
| 1.3C | 0.10 | 58.86 | 104 | 25.5 |
| 1.4C | 1.00 | 54.06 | 93 | 15.2 |

Control, Screened wet (No Sucralose)

| | | | |
|---|---|---|---|
| 1D | 0.00 | 74.30 | 181 |

Sucralose Added to water, Screened wet

| | | | | |
|---|---|---|---|---|
| 1.1E | 0.01 | 82.71 | 226 | 11.3 |
| 1.2E | 5.00 | 85.06 | 218 | 14.5 |

Sucralose to the bowl dry, Screened wet

| | | | | |
|---|---|---|---|---|
| 1.1F | 0.01 | 77.05 | 205 | 3.7 |
| 1.2F | 5.00 | 77.41 | 190 | 4.2 |

TABLE 2

| | Sample (% Retained) | | | | | | | | |
|---|---|---|---|---|---|---|---|---|---|
| Sieve | 1A | 1.1B | 1.2B | 1.3B | 1.4B | 1.1C | 1.2C | 1.3C | 1.4C |
| 80 | 23.25 | 24.52 | 31.32 | 31.87 | 32.47 | 29.55 | 32.90 | 34.08 | 32.70 |
| 120 | 6.49 | 8.94 | 8.92 | 8.76 | 7.59 | 6.63 | 7.34 | 8.13 | 7.82 |
| 170 | 10.48 | 14.57 | 12.29 | 10.86 | 10.69 | 11.36 | 10.87 | 10.48 | 8.53 |
| 200 | 6.69 | 10.55 | 7.43 | 6.67 | 6.59 | 7.24 | 6.54 | 6.17 | 5.02 |
| 325 | 19.86 | 25.53 | 19.03 | 21.71 | 20.28 | 22.71 | 18.51 | 18.32 | 16.85 |
| 400 | 7.78 | 7.14 | 6.34 | 7.87 | 6.69 | 7.14 | 5.33 | 6.76 | 7.22 |
| PAN | 25.45 | 8.74 | 14.67 | 12.25 | 15.68 | 15.38 | 18.51 | 16.06 | 21.87 |
| MEAN | 73 | 95 | 102 | 102 | 100 | 93 | 99 | 104 | 93 |
| STD | 3.01 | 2.11 | 2.72 | 2.60 | 2.86 | 2.66 | 3.11 | 3.02 | 3.60 |

TABLE 3

| | Sample (% Retained) | | | | |
|---|---|---|---|---|---|
| Sieve | 1D | 1.1E | 1.2E | 1.1F | 1.2F |
| 40 | 27.91 | 32.76 | 28.89 | 30.26 | 28.57 |
| 60 | 14.26 | 14.05 | 14.84 | 15.33 | 13.37 |
| 80 | 7.23 | 7.68 | 10.83 | 7.62 | 8.21 |
| 120 | 9.04 | 10.82 | 13.54 | 9.22 | 10.03 |
| 170 | 9.94 | 11.63 | 12.34 | 9.32 | 11.04 |
| 200 | 5.92 | 5.76 | 4.61 | 5.31 | 6.18 |
| PAN | 25.70 | 17.29 | 14.94 | 22.95 | 22.59 |
| MEAN | 181 | 226 | 218 | 205 | 190 |
| STD | 4.22 | 3.55 | 2.93 | 4.11 | 3.81 |

### Part B: AVICEL® pH 102 Batches

### Sample 3A (Dry Screened):

As a control, 255.7 g of purified water was slowly added manually to 350 g AVICEL® over 25-35 minutes while mixing in a 2-quart Hobart mixer. The mixture was dried at 50° C. for 24 hours and screened though a 20 mesh screen.

### Samples 3.1B, 3.2B, 3.3B, 3.4B (Dry Screened):

0.01, 0.05, 0.1, and 1% of sucralose respectively was prepared as four solutions in 255.7 g of water per solution. Each granulation sample was prepared by slowly and manually adding the individual sucralose solutions to 350 g of AVICEL® while mixing over 25-35 minutes in a 2-quart Hobart mixer. The samples were dried at 50° C. for 24 hours and screened though a 20 mesh screen.

### Samples 3.1C, 3.2C, 3.3C, 3.4C (Dry Screened):

0.01, 0.05, 0.1, and 1% of sucralose respectively was blended individually with 350 g of AVICEL® each as a dry mixture in a 2-quart Hobart mixing bowl. 255.7 g of water was slowly and manually added to each sample while mixing over 25-35 minutes in a 2-quart Hobart mixer. The samples were dried at 50° C. for 24 hours and screened though a 20 mesh screen.

### Sample 3D (Wet Screened):

As a control, 0.0% sucralose was added and 255.7 g of purified water was slowly added manually to 350 g AVICEL over 25-35 minutes while mixing in a 2-quart Hobart mixer. The mixture was screened though a 20 mesh screen, and then dried at 50° C. for 24 hours.

### Samples 3.1E, 3.2E (Wet Screened):

0.01% and 5% of sucralose respectively was prepared as two solutions in 255.7 g of purified water per solution. Each granulation sample was prepared by slowly and manually adding the individual sucralose solutions to 350 g of AVICEL® while mixing over 25-35 minutes in a 2-quart Hobart mixer. The samples were screened though a 20 mesh screen and then dried at 50° C. for 24 hours.

### Samples 3.1F, 3.2F (Wet Screened):

0.01 and 5% of sucralose respectively was blended individually with 350 g of AVICEL® each as a dry mixture in a 2-quart Hobart mixing bowl. 255.7 g of purified water was slowly and manually added to each sample while mixing over 25-35 minutes in a 2-quart Hobart mixer. The samples were screened though a 20 mesh screen and dried at 50° C. for 24 hours.

The particle size results for batches produced in Part A (using AVICEL® pH 102) are displayed in Table 4. Particle size was analyzed via sieve cut analysis, using an ATM Sonic Sifter and approximately 10 g of granulation. The amounts of material retained on each sieve cut are displayed in Table 5 and Table 6. The results demonstrate that the addition of sucralose to the solution and in the dry blend results in a substantial increase in particle size, demonstrating the binding effect. The range of particle size increase for particles greater than 74 microns (200 mesh) was between 0.9 and 8.7%. These results are evident both in wet screening and screening of the material following a drying step.

TABLE 4

| | | | | |
|---|---|---|---|---|
| | | | | >74 |
| | % | % >74 | Mean Particle | microns |
| SAMPLE | SUCRALOSE | microns | Size (microns) | % Increase |

Control, Screened dry (No Sucralose)

| | | | |
|---|---|---|---|
| 3A | 0.00 | 80.75 | 129 |

Sucralose Added to water, Screened dry

| | | | | |
|---|---|---|---|---|
| 3.1B | 0.01 | 85.11 | 137 | 5.4 |
| 3.2B | 0.05 | 82.35 | 132 | 2.0 |
| 3.3B | 0.10 | 87.79 | 143 | 8.7 |
| 3.4B | 1.00 | 86.76 | 145 | 7.4 |

Sucralose to the bowl dry, Screened dry

| | | | | |
|---|---|---|---|---|
| 3.1C | 0.01 | 83.65 | 135 | 3.6 |
| 3.2C | 0.05 | 86.17 | 140 | 6.7 |
| 3.3C | 0.10 | 83.05 | 139 | 2.8 |
| 3.4C | 1.00 | 84.88 | 140 | 5.1 |

Control, Screened wet (No Sucralose)

| | | | |
|---|---|---|---|
| 3D | 0.00 | 89.68 | 206 |

Sucralose Added to water, Screened wet

| | | | | |
|---|---|---|---|---|
| 3.1E | 0.01 | 91.65 | 177 | 2.2 |
| 3.2E | 5.00 | 94.47 | 214 | 5.3 |

Sucralose to the bowl dry, Screened wet

| | | | | |
|---|---|---|---|---|
| 3.1F | 0.01 | 90.46 | 193 | 0.9 |
| 3.2F | 5.00 | 93.99 | 209 | 4.8 |

TABLE 5

| | Sample (% retained) | | | | | | | | |
|---|---|---|---|---|---|---|---|---|---|
| Sieve | 3A | 3.1B | 3.2B | 3.3B | 3.4B | 3.1C | 3.2C | 3.3C | 3.4C |
| 80 | 27.42 | 30.40 | 29.19 | 32.09 | 35.21 | 29.89 | 32.44 | 32.80 | 31.34 |
| 120 | 26.41 | 27.96 | 26.28 | 29.77 | 26.38 | 27.18 | 27.36 | 25.77 | 28.16 |
| 170 | 19.25 | 19.76 | 19.56 | 19.58 | 18.25 | 18.96 | 19.30 | 17.84 | 18.51 |
| 200 | 7.66 | 6.99 | 7.32 | 6.36 | 6.92 | 7.62 | 7.06 | 6.64 | 6.87 |
| 325 | 14.82 | 12.16 | 13.94 | 10.49 | 10.93 | 13.14 | 11.44 | 12.78 | 11.14 |
| 400 | 1.92 | 1.72 | 2.21 | 1.11 | 1.50 | 1.71 | 1.69 | 2.08 | 2.09 |
| PAN | 2.52 | 1.01 | 1.50 | 0.61 | 0.80 | 1.50 | 0.70 | 2.08 | 1.89 |
| MEAN | 129 | 137 | 132 | 143 | 145 | 135 | 140 | 139 | 140 |
| STD | 1.85 | 1.76 | 1.80 | 1.71 | 1.77 | 1.80 | 1.74 | 1.90 | 1.87 |

TABLE 6

| | Sample (% Retained) | | | | |
|---|---|---|---|---|---|
| Sieve | 3D | 3.1E | 3.2E | 3.1F | 3.2F |
| 40 | 24.35 | 12.17 | 19.92 | 18.17 | 19.24 |
| 60 | 13.13 | 13.98 | 15.90 | 14.42 | 15.53 |
| 80 | 13.43 | 17.51 | 20.82 | 17.06 | 19.54 |
| 120 | 18.94 | 25.55 | 20.62 | 20.71 | 21.54 |
| 170 | 14.23 | 16.70 | 13.08 | 14.62 | 13.63 |
| 200 | 5.61 | 5.73 | 4.12 | 5.48 | 4.51 |
| PAN | 10.32 | 8.35 | 5.53 | 9.54 | 6.01 |
| MEAN | 206 | 177 | 214 | 193 | 209 |
| STD | 2.37 | 1.94 | 1.99 | 2.14 | 2.00 |

### Example 4

### Sweetness Evaluation/Study

A sample of a granulation composition of the invention (i.e., containing sucralose, a wettable material and an active agent) and a control sample of a granulation made without sucralose, are both ingested by ten (10) participants. On average, the participants did not perceive any sweetness due to the sucralose used in granulating the inventive sample.

The examples provided herein further illustrate the compositions and methods of the present invention. These examples are illustrative only and are not intended to limit the scope of the invention in any way.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed:

1. A method of making a granulation comprising the steps of:
(a) combining sucralose, a polar solvent, a wettable material and an active agent, thereby forming a mixture; and
(b) drying the mixture, thereby forming the granulation.

2. The method of claim 1, wherein the amount of sucralose present in the granulation is about 0.01 wt. % to about 5 wt. % based on the total weight of the granulation.

3. The method of claim 1, wherein the active agent and sucralose are present in a ratio of about 6.25:0.005 to about 6.25:0.05 active agent to sucralose.

4. The method of claim 1, wherein the granulation has a moisture content of about 0.1% to about 5% by weight.

5. The method of claim 1, wherein the active agent is a pharmaceutical active agent selected from the group consisting of diphenhydramine, pseudoephedrine, chlorpheniramine, cetirizine, loperamide and mixtures thereof.

6. The method of claim 1, wherein the sucralose is combined with the polar solvent.

7. The method of claim 1, wherein the sucralose is in dry form.

8. The method of claim 1, wherein the active agent is combined with the sucralose and polar solvent, and layered onto the wettable material.

9. The method of claim 1, further comprising the step of coating the granulation with a taste masking system.

10. The method of claim 1, wherein the polar solvent is water.

11. The method of claim 1, wherein the wettable material is selected from the group consisting of sucrose, mannitol, dextrose, lactose, lactitol, sorbitol, silicified microcrystalline cellulose, microcrystalline cellulose, and mixtures thereof.

12. A method of increasing the mean particle size of a mixture of sucralose, a wettable material and an active agent comprising the steps of:
combining sucralose, a polar solvent, a wettable material and an active agent, thereby forming a mixture; and
drying the mixture and removing the polar solvent, thereby forming a granulation comprising a plurality of granules,
wherein the mean particle size of the granulation is at least about 1.0% greater than the mean particle size of the active agent, wettable material and sucralose.

13. The method of claim 12, wherein the average particle size of the granulation is at least about 2% greater than the average particle size of the mixture of the active agent, wettable material and sucralose as measured through an 18 mesh screen and on a 200 mesh screen.

14. The method of claim 12, wherein the sucralose has a concentration of about 0.01% to about 5% by weight based on the combined weight of the sucralose, the active agent and the wettable material after drying.

15. The method of claim 12, wherein the granulation is further blended with a matrix and compressed into a chewable tablet.

16. The method of claim 15, wherein the granulation is less than about 10 percent by weight of the chewable tablet.

17. The method of claim 12, wherein the granulation is further coated with a polymer coating.

18. A method of making a granulation comprising the steps of:
(a) coating/layering a wettable material with a solution or suspension comprising sucralose, a polar solvent, and an active agent, thereby forming a mixture; and
(b) drying the mixture, thereby forming the granulation.

19. A method of making a granulation comprising the steps of:
(a) combining sucralose, a polar solvent, a wettable material and an active agent, thereby forming a mixture; and
(b) drying the mixture, thereby forming the granulation,
wherein the granulation exhibits an increase in mean particle size of at least about 1% when compared to a substantially similar granulation composition absent the sucralose.
